(19) United States
(12) Patent Application Publication
(10) Pub. No.: US 2021/0277778 A1
(43) Pub. Date: Sep. 9, 2021

(54) METHOD FOR EVALUATING BRITTLENESS OF DEEP SHALE RESERVOIR AND COMPUTER READABLE STORAGE MEDIUM

(71) Applicant: CHINA UNIVERSITY OF PETROLEUM, Qingdao City (CN)

(72) Inventors: Xingyao YIN, Qingdao City (CN); Zhengqian MA, Qingdao City (CN); Zhaoyun ZONG, Qingdao City (CN); Guochen WU, Qingdao City (CN); Guangzhi ZHANG, Qingdao City (CN); Danping CAO, Qingdao City (CN); Fanchang ZHANG, Qingdao City (CN); Kai LIANG, Qingdao City (CN); Jiajia ZHANG, Qingdao City (CN); Kun LI, Qingdao City (CN)

(73) Assignee: CHINA UNIVERSITY OF PETROLEUM, Qingdao City (CN)

(21) Appl. No.: 16/945,372

(22) Filed: Jul. 31, 2020

(30) Foreign Application Priority Data
Mar. 4, 2020 (CN) 202010142586.0

(51) Int. Cl. E21B 49/08; G01N 33/24; G01V 1/50

(52) U.S. Cl. CPC E21B 49/087; G01N 33/24; G01V 2210/6169; G01V 2210/6242; G01V 1/50

(57) ABSTRACT

A method for evaluating brittleness of a deep shale reservoir and a computer readable storage medium. The method includes determining a Rickman brittleness index of the deep shale reservoir and determining an effective pressure of the deep shale reservoir according to a pore pressure and an overlying formation pressure of the deep shale reservoir. The Rickman brittleness index is adjusted to obtain the brittleness index of the deep shale reservoir according to an exponential relationship of the brittleness index with the effective pressure of the deep shale reservoir. Inherent properties, such as rock brittle mineral content and the like, are better indicated by the Rickamn brittleness index, and then the brittleness index of the deep shale reservoir is obtained by utilizing the exponential relationship of the brittleness index with the effective pressure of the deep shale reservoir, to realize reasonable evaluation for the brittleness of the deep shale reservoir.

### TECHNICAL FIELD

The invention relates to the field of oil and gas exploration and development, in particular to a method for evaluating brittleness of a deep shale reservoir and a computer readable storage medium.

### BACKGROUND ART

Shale oil gas resources are rich in the world, regarded as alternative energy for conventional oil and gas, and the deep shale reservoir become increasingly a hot topic of geophysicists due to its significant oil gas reserves prospects. Brittleness evaluation, which is closely related to the design of fracturing reform and development scheme, has become a major problem in the field of oil and gas exploration and development.

At present, the reservoir brittleness evaluation methods at home and abroad mainly comprise four types: (1) measuring mineral content in a laboratory to characterize brittleness (i.e., mineral brittleness index); (2) characterizing the rock brittleness by elastic mechanical parameters obtained by a geophysical method and a combination thereof; (3) carrying out rock mechanics experiments in a laboratory, and performing brittleness evaluation by stress-strain characteristics; (4) making a study based on conventional fracturing experimental data. The seismic evaluation technology of reservoir brittleness mainly uses the second method: characterizing the rock brittleness by elastic mechanical parameters and a combination thereof. The evaluation method based on the elastic parameters mainly uses Young's modulus and Poisson's ratio, or Lame parameters to evaluate the shale brittleness. The Young's modulus is the ratio of longitudinal stress to strain, which reflects the stiffness of rock; the Poisson's ratio is the ratio of transverse strain to longitudinal strain, which reflects the plasticity of rock. The Young's modulus and Poisson's ratio are comprehensive responses of material composition, structure, porosity and fluid in rocks under certain environment, while the elastic information of strata can be obtained by logging, seismic and other means so as to reflect the comprehensive responses of internal characteristics of strata under the action of in-situ environment. The Lame parameters are similar to the Young's modulus and Poisson's ratio, which comprehensively reflect the composition and structural characteristics of underground strata. Specifically, Rickman (2008) proposed a brittleness index based on normalized Young's modulus and Poisson's ratio by statistical analysis of Barnnet shale in the United States:

$$\begin{matrix}E\_BRIT = \frac{( E - 1 )}{7} \times 100 , & ( 1 ) \\ σ\_BRIT = \frac{( σ - 0.4 )}{0.15 - 0.4} \times 100 , & ( 2 ) \\ {BI}_{Rickman} = \frac{E\_BRIT + σ\_BRIT}{2} , & ( 3 )\end{matrix}$$

wherein, E_BRIT, σ_BRIT and BI_{Rickman} are normalized Young's modulus, Poisson's ratio brittleness index and Rickman brittleness index respectively, and E and σ are the Young's modulus and Poisson's ratio of an observation point respectively. Rickman believed that high brittleness exhibited high Young's modulus and low Poisson's ratio. They are then generalized to obtain a general Rickman brittleness index:

$$B {I}_{Rickman} = \frac{1}{2} ( \frac{E - {E}_{m i n}}{{E}_{m ax} - {E}_{m i n}} + \frac{σ - {σ}_{ma x }}{{σ}_{m i n} - {σ}_{ma x}} ) , \qquad ( 4 )$$

wherein, Eₘₐₓ, Eₘᵢₙ, σₘₐₓ, and αₘᵢₙ respectively represent a maximum Young's modulus, a minimum Young's modulus, a maximum Poisson's ratio and a minimum Poisson's ratio of a target working area.

Furthermore, Goodway (2010) used Lame parameters and shear modulus to characterize shale brittleness, wherein λ and μ represent the Lame parameter set.

Guo (2013) proposed a corresponding physical model to characterize the brittleness of shale by the ratio of Young's modulus to Poisson's ratio BI_{Guo}=E/σ.

Liu Zhishui (2015) proposed a calculation expression

$$B {I}_{Liu} = ( \frac{E - {E}_{m i n}}{{E}_{m ax} - {E}_{m in}} ) / ( \frac{σ - {σ}_{m i n}}{{σ}_{m ax} - {σ}_{m i n}} )$$

of rock brittleness based on normalized elastic parameters.

However, the inventors of the present application have found that the main research object of the existing seismic evaluation technology of shale reservoir brittleness is a shallow shale target area. If it is directly used for deep shale target processing, an inaccurate problem exists.

### SUMMARY OF THE INVENTION

In order to solve the technical problem or at least partially solve the technical problem, the application provides a method for evaluating brittleness of a deep shale reservoir and a computer readable storage medium.

In a first aspect, the invention provides a method for evaluating brittleness index of a deep shale reservoir BI, characterized by comprising the steps of: determining a Rickman brittleness index BI_{Rickman} of the deep shale reservoir; determining an effective pressure Pe of the deep shale reservoir according to a pore pressure Pₚ and an overlying formation pressure P of the deep shale reservoir; and adjusting BI_{Rickman} to obtain BI according to an exponential relationship of the brittleness index BI with Pe of the deep shale reservoir.

In some embodiments, the adjusting BI_{Rickman} to obtain BI according to an exponential relationship of the brittleness index BI with Pe of the deep shale reservoir comprises determining BI as follows: BI=BI_{Rickman} [e^{m(40-Pe)}−n]/l; wherein, l is amplitude modulation factor, and m and n are obtained by fitting laboratory rock core stress-strain analysis data.

In some embodiments, the determining an effective pressure Pe of the deep shale reservoir according to a pore pressure Pₚ and an overlying formation pressure P of the deep shale reservoir comprises: determining the overlying formation pressure P of the deep shale reservoir according to P=∫ₜ_{₀}^{t}ρ(t)V(t)gdt, wherein g is gravitational acceleration, ρ(t) is measured rock density, V(t) is measured rock velocity, t is time depth of the deep shale reservoir, and t₀ is time depth of a reference datum plane; determining a hydrostatic pressure P₀ of a deep shale reservoir according to P₀=ρ_{w}gH, wherein g is gravitational acceleration, H is formation burial depth, and ρ_{w} is formation water density; determining a pore pressure Pₚ of the deep shale reservoir according to the Eaton method, wherein,

$${P}_{p} = P - ( P - {P}_{0} ) {( \frac{Δ {t}_{n}}{Δ {t}_{s}} )}^{c} ,$$

c is the Eaton coefficient, Δtₘ is measured interval transit time, and Δtₙ is normal compaction interval transit time; and determining the effective pressure Pe of the deep shale reservoir according to Pe=P−Pₚ.

In certain embodiments, it further comprises:

determining Δtₙ according to Δtₙ=Δtₘ+(Δtₘₗ−Δtₘ)e^{-aH}, wherein Δtₘ is the interval transit time of rock matrix, Δtₘₗ is the interval transit time of the surface or seabed, H is the stratum buried depth, and a is a regional index.

In a second aspect, the present application provides a computer device comprising a memory, a processor, and a computer program stored on the memory and being operable on the processor; and the computer program, when executed by the processor, performs the steps of the method for evaluating the brittleness of the deep shale reservoir.

In a third aspect, the present application provides a computer readable storage medium having stored thereon a brittleness evaluation program for a deep shale reservoir which, when executed by a processor, performs the steps of the method for evaluating the brittleness of the deep shale reservoir.

Compared with the prior art, the technical solution provided by the embodiment of the invention has the following advantages: according to the method provided by the embodiment of the invention, inherent properties such as rock brittle mineral content and the like are better indicated by the Rickamn brittleness index, and the brittleness index of the deep shale reservoir is obtained by utilizing the exponential relationship of the brittleness index with the effective pressure of the deep shale reservoir, so as to realize effective evaluation of the brittleness of the deep shale reservoir, causing a prediction result of the brittleness of the deep shale reservoir more reasonable.

### BRIEF DESCRIPTION OF THE DRAWINGS

The accompanying drawings, which are incorporated in and constitute a part of this specification, illustrate embodiments consistent with the present application and, together with the description, serve to explain the principles of the application.

In order to more clearly illustrate the embodiments of the present application or the technical solutions in the prior art, reference will now be made briefly to the accompanying drawings, which are used in the description of the embodiments or the prior art, and it will be obvious to a person skilled in the art that other drawings may be obtained from these drawings without involving any inventive effort.

FIG. 1 is a crossplot of brittle mineral contents (i.e., brittle mineral index) with Rickman brittleness index of a deep shale reservoir in a working area;

FIG. 2 is a schematic diagram showing the relationship of rock brittleness index with effective pressure of a deep shale reservoir in a working area;

FIG. 3 is a flow chart of an implementation of a method for evaluating brittleness of a deep shale reservoir provided by an embodiment of the present application;

FIG. 4a is a Rickman brittleness index prediction result of a deep shale reservoir in a working area;

FIG. 4b is a brittle index prediction result incorporating effective pressure of a deep shale reservoir in a working area;

FIG. 5 is a hardware schematic diagram of an implementation of a computer device provided by an embodiment of the present application.

### DETAILED DESCRIPTION

It should be understood that the specific embodiments described herein are merely illustrative of and not intended to limit the present application.

In the following description, suffixes such as “module”, “component”, or “unit” used to represent elements are used merely to facilitate the description of the present application and have no particular meaning in itself. Thus, “module”, “component”, or “unit” may be used in combination.

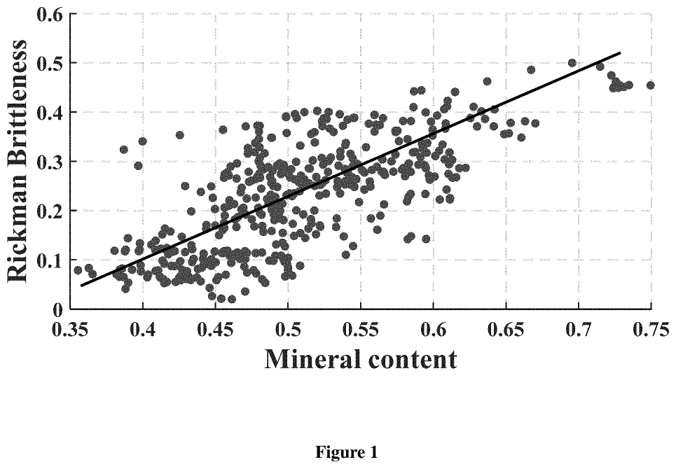

FIG. 1 is a crossplot of brittle mineral contents with Rickman brittleness index of a deep shale reservoir in a working area, the degree of dispersion of which is affected by non-mineral inherent properties such as rock cracks. Referring to FIG. 1, Rickamn brittleness index has a good mapping relationship with brittle mineral contents, i.e., the Rickamn brittleness index is a good indicator of rock brittle mineral content without changing other rock conditions. Thus, in an embodiment of the present application, the effects of rock intrinsic properties (e.g., brittle mineral content) and external factors (e.g., effective pressure of formation) on the brittle characteristics of deep shale reservoirs are considered.

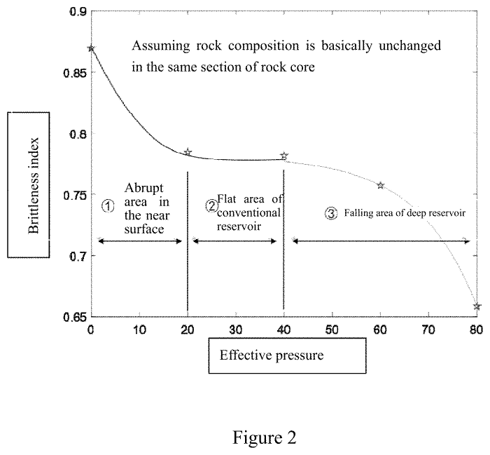

FIG. 2 shows the relationship between rock brittleness and effective pressure calculated by laboratory stress-strain analysis of a rock core sample from the same section of the deep shale reservoir under different effective pressures. As can be seen from FIG. 2, the relationship curve is roughly divided into the following three sections.

\textcircled{1} A shallow surface layer abrupt area: this area simulates effective pressure environment of a shallow formation.

\textcircled{2} A conventional reservoir flat area: this area simulates relationship of the brittleness with the effective pressure of the shale reservoir in the conventional shallow part, and it is found that effective pressure has little effect on rock brittleness.

\textcircled{3} A deep reservoir falling area: this area simulates relationship between the brittleness with the effective pressure of the deep shale reservoir.

The inventors of the present application have found that the brittleness of the deep shale reservoir is exponentially related to the effective pressure. In an embodiment of the invention, the brittleness influencing factors of the deep shale reservoir are integrated, and the brittleness index of the deep shale reservoir based on the effective pressure is constructed as follows:

$$BI = {BI}_{Rickman} [ {e}^{m ( 40 - Pe )} - n ] / l = \frac{1}{2} ( \frac{E - {E}_{m i n}}{{E}_{ma x} - {E}_{m i n}} + \frac{σ - {σ}_{ma x}}{{σ}_{m i n} - {σ}_{m a x}} ) \frac{[ {e}^{m ( 40 - Pe )} - n ]}{l} \qquad ( 5 )$$

wherein, BI_{Rickman} is Rickman brittleness index (see Formula 4) for characterizing inherent properties such as the brittle mineral content; l is amplitude modulation factor, and if only the relative change of the brittleness of the working area is analyzed, the parameter can be set to 1; m and n can be obtained by fitting laboratory rock core stress-strain analysis data; for example, the data fitting result of FIG. 2 is m=−0.06982, n=−98.4.

In an embodiment of the application, the pore pressure Pₚ and the overlying formation pressure P in the formula (5) are estimated by using seismic data, and Young's modulus E, Poisson's ratio σ, density ρ and velocity V of a target area are inverted, so that the deep shale reservoir brittleness evaluation is realized.

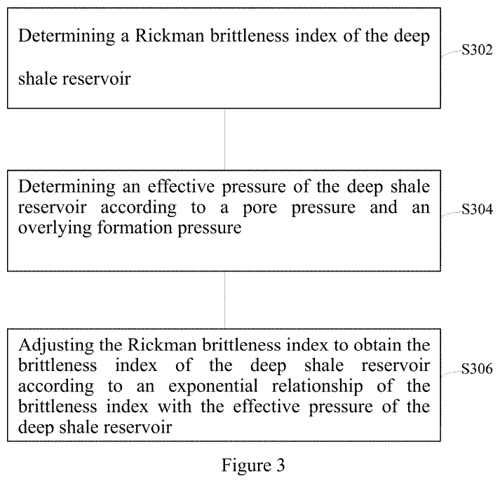

FIG. 3 is a flow chart of an implementation of a method for evaluating brittleness of a deep shale reservoir provided by an embodiment of the present application. As shown in FIG. 3, the method includes steps S302 to S306.

Step S302, determining a Rickman brittleness index of the deep shale reservoir.

In certain embodiments, the Rickman brittleness index is determined as follows.

First step, elastic impedance inversion. Under the prior constraint of the model, a maximum posterior probability solution of target inversion is derived based on the Bayesian theory:

(G^{T}G+μQ+αC)m=(G^{T}d+C^{T}ξ) (6)

wherein, G is relation matrix between reflection coefficients m and seismic data d; μ and α are constraint coefficients and can be given with corresponding constant values according to the target characteristic; ξ=ln EI(t)/ln EI(t₀) provides an elastic impedance model constraint for inversion, EI(t) is an elastic impedance value at a moment t, and t₀ is initial moment;

ξ=Cm, C is the integral matrix;

$$Q = diag ( \frac{1}{{( 1 + {m}_{1}^{2} / {σ}_{m}^{2} )}^{2}} , \frac{1}{{( 1 + {m}_{2}^{2} / {σ}_{m}^{2} )}^{2}} , … , \frac{1}{{( 1 + {m}_{n}^{2} / {σ}_{m}^{2} )}^{2}} ) ,$$

σₘ is the parameter of the Cauchy distribution model obeyed by the target parameters.

The reflection coefficient m can be obtained by solving Formula (6) by using the iterative reweighted least square method, and then the elastic impedance is obtained by using the trace integration idea:

EI=EI(t₀)exp[2∫ₜ_{₀}^{t}m(τ)dτ] (7)

Step two, extracting reservoir elastic parameters based on the elastic impedance inversion data volume; and establishing the functional relationship between the Young's modulus and the elastic impedance:

$$[ \begin{matrix}ln ( \frac{EI ( {θ}_{1} , {t}_{1} )}{{EI}_{0}} ) & ln ( \frac{EI ( {θ}_{2} , {t}_{1} )}{{EI}_{0}} ) & ln ( \frac{EI ( {θ}_{3} , {t}_{1} )}{{EI}_{0}} ) \\ ln ( \frac{EI ( {θ}_{1} , {t}_{2} )}{{EI}_{0}} ) & ln ( \frac{EI ( {θ}_{2} , {t}_{2} )}{{EI}_{0}} ) & ln ( \frac{EI ( {θ}_{3} , {t}_{2} )}{{EI}_{0}} ) \\ … & … & … \\ ln ( \frac{EI ( {θ}_{1} , {t}_{n} )}{{EI}_{0}} ) & ln ( \frac{EI ( {θ}_{2} , {t}_{n} )}{{EI}_{0}} ) & ln ( \frac{EI ( {θ}_{3} , {t}_{n} )}{{EI}_{0}} )\end{matrix} ] [ \begin{matrix}a ( {θ}_{1} ) \\ a ( {θ}_{2} ) \\ a ( {θ}_{3} )\end{matrix} ] = [ \begin{matrix}ln ( \frac{E ( {t}_{1} )}{{E}_{0}} ) \\ ln ( \frac{E ( {t}_{2} )}{{E}_{0}} ) \\ … \\ ln ( \frac{E ( {t}_{n} )}{{E}_{0}} )\end{matrix} ] \qquad ( 8 )$$

wherein EI(θᵢ,t) represents the elastic impedance value at a t tⱼ^{th} moment when an incident angle is θᵢ; the elastic impedance inversion data volume of the near-well seismic channel and the measured value of the Young's modulus from the well log are substituted into Formula (8) to fit a reasonable coefficient vector [a(θ₁) a(θ₂) a(θ₃)]^{T}, and then the elastic impedance inversion data volume of the target working area and the fitted coefficient vector are substituted into Formula (8) to obtain the Young's modulus data volume of the target working area. For Poisson's ratio σ, rock density ρ and velocity V in the target working area are extracted with the same steps.

Step S304, determining an effective pressure of the deep shale reservoir according to a pore pressure and an overlying formation pressure of the deep shale reservoir.

In some embodiments, the effective pressure is determined as follows.

Firstly, the overlying formation pressure is calculated:

P=∫ₜ_{₀}^{t}ρ(t)V(t)gdt (9)

wherein, g is gravitational acceleration in the unit of m/s²; ρ(t) is measured rock density in the unit of kg/m³; V(t) is measured rock velocity in the unit of m/s; and t is time depth of the deep shale reservoir, and t₀ is time depth of a reference datum plane.

Then, the hydrostatic pressure P₀ is calculated:

P₀=ρ_{w}gH (10)

wherein, H is formation buried depth in the unit of m; and ρ_{w} is the formation water density in the unit of kg/m³.

Next, the pore pressure Pₚ is estimated using the Eaton's method:

$${P}_{p} = P - ( P - {P}_{0} ) {( \frac{Δ {t}_{n}}{Δ {t}_{s}} )}^{c} \qquad ( 11 )$$

wherein, c is the Eaton coefficient, an applicable constant value is selected according to the characteristics of the working area, and c=3 is generally applicable to under-compaction overpressure; Δtₛ=1/V is measured interval transit time in the unit of μs/m; and Δtₙ is normal compaction interval transit time in the unit of μs/m and can be calculated by Formula (12):

Δtₙ,=Δtₘ+(Δtₘₗ−Δtₘ)e^{-aH} (12)

wherein, Δtₘ is interval transit time of a rock matrix in the unit of μs/m, and Δtₘ can be estimated according to mineral well logging data and a rock physical modeling theory; Δtₘ is interval transit time of the surface or seabed in the unit of μs/m; H is formation buried depth in the unit of m; and the constant a is a regional index and can be given empirically.

Finally, Formulas (9)-(12) are integrated, and the inverted rock density and velocity data volume in the working area are substituted to estimate the effective pressure Pe=P−P of the formation in the working area.

Step S306, adjusting the Rickman brittleness index to obtain the brittleness index of the deep shale reservoir according to an exponential relationship of the brittleness index with the effective pressure of the deep shale reservoir.

In some embodiments, according to Formula (5), a brittleness index of the deep shale reservoir is obtained, thereby achieving a deep reservoir brittleness evaluation in the working area.

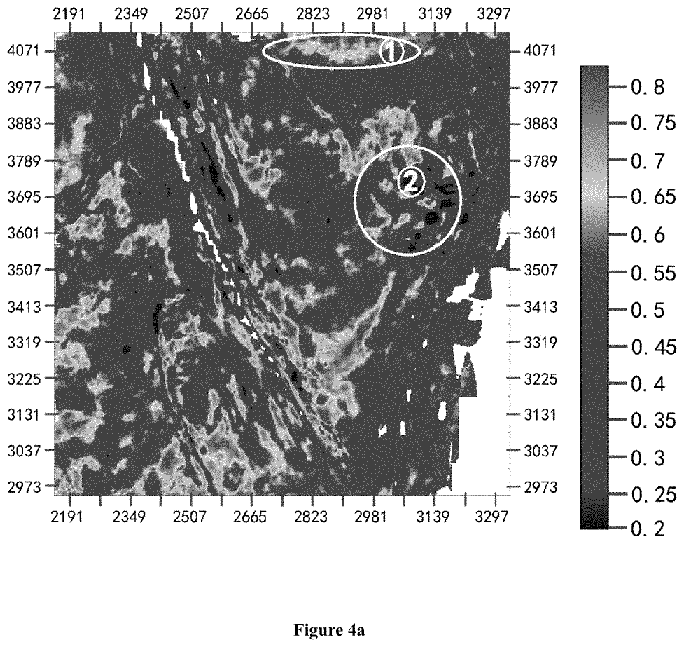

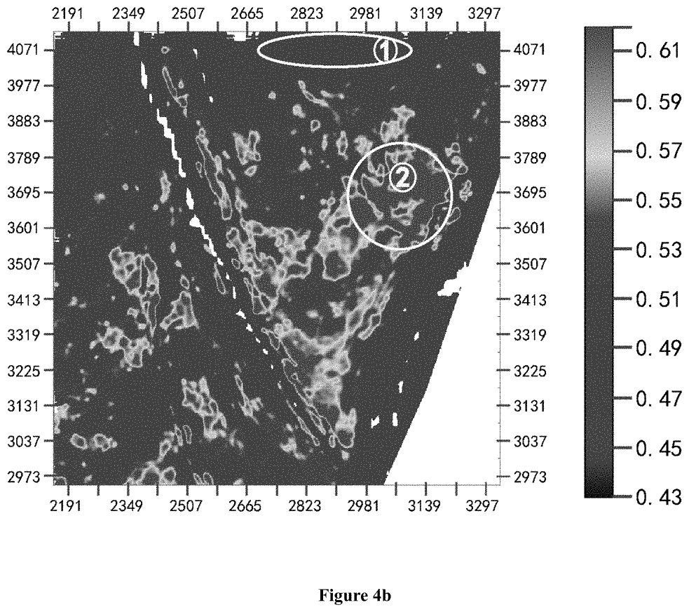

FIG. 4a shows the seismic prediction result of the Rickman brittleness index of a horizon slice of the deep shale reservoir in the working area, and FIG. 4b shows the prediction result of the brittleness index considering the effective pressure in the working area.

In the figures, the buried depth of Area \textcircled{1} is greater than that of Area \textcircled{2}, and the brittleness of Area \textcircled{2} should be stronger than that of Area \textcircled{1}. The analysis and prediction results show that the brittleness index considering the effective pressure proposed by the application is more reasonable for the deep shale reservoir.

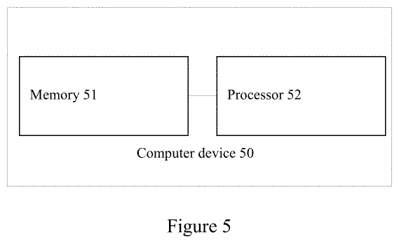

The embodiment of the application also provides a computer device, such as a smart phone, a tablet computer, a notebook computer, a desktop computer, a rack server, a blade server, a tower server or a cabinet server (comprising an independent server or a server cluster formed by a plurality of servers), and the like which can execute programs. As shown in FIG. 5, the computer device 50 of the present embodiment includes at least, but is not limited to a memory 51 and a processor 52 communicatively connected to each other via a system bus. It should be noted, however, that it should be understood that not all illustrated components may be required and that more or fewer components may be implemented instead.

In this embodiment, the memory 51 (i.e., readable storage medium) includes flash memory, hard disk, multimedia card, card-type memory (e.g., SD or DX memory, etc.), random access memory (RAM), static random access memory (SRAM), read-only memory (ROM), electrically erasable programmable read-only memory (EEPROM), programmable read-only memory (PROM), magnetic memory, magnetic disk, optical disk, etc. In some embodiments, the memory 51 may be an internal storage unit of the computer device 50, such as a hard disk or memory of the computer device 50. In other embodiments, the memory 51 may also be an external storage device of the computer device 50, such as a plug-in hard disk, a Smart Media Card (SMC), a Secure Digital (SD) card, a Flash Card, etc., provided on the computer device 50. Of course, the memory 51 may also comprise both the internal storage unit and the external storage device of the computer device 50. In this embodiment, the memory 51 is typically used to store an operating system and various types of application software installed on the computer device 50, such as program codes for deep shale reservoir brittleness evaluation, etc. In addition, the memory 51 may also be used to temporarily store various types of data that have been or are to be output.

The processor 52 may be, in some embodiments, a central processing unit (CPU), a controller, a microcontroller, a microprocessor, or other data processing chip. The processor 52 is typically used to control the overall operation of the computer device 50. In this embodiment, the processor 52 is configured to run program codes or process data stored in the memory 51, such as a brittleness evaluation program for a deep shale reservoir, to implement the steps of the method for evaluating brittleness of the deep shale reservoir.

This embodiment also provides a computer-readable storage medium, such as flash memory, hard disk, multimedia card, card-type memory (e.g., SD or DX memory, etc.), random access memory (RAM), static random access memory (SRAM), read-only memory (ROM), electrically erasable programmable read-only memory (EEPROM), programmable read-only memory (PROM), magnetic storage, magnetic disk, optical disk, server, App application mall or the like which has stored thereon a computer program, and the computer program, when executed by a processor, performs a corresponding function. The computer readable storage medium of the embodiment is used for storing a brittleness evaluation program for a deep shale reservoir which, when executed by a processor, realizes a method for evaluating brittleness of the deep shale reservoir.

It should be noted that the terms “including”, comprising”, or any other variation thereof, are intended to cover a non-exclusive inclusion, such that a process, method, article, or apparatus that includes a list of elements does not include only those elements but may include other elements not expressly listed or inherent to such process, method, article, or apparatus. An element defined by the phrase “comprises a . . . ” does not, without more constraints, preclude the existence of additional identical elements in the process, method, article, or apparatus that comprises the element.

The above-mentioned embodiments of the present application are by way of illustration only and do not represent advantages or disadvantages of the embodiments.

From the above description of the embodiments, it will be clear to a person skilled in the art that the above-described embodiment method can be implemented by means of software plus a necessary general-purpose hardware platform, but in many cases the former is a better embodiment. Based on this understanding, the technical solution of the present application, in essence or in part contributing to the prior art, may be embodied in the form of a software product stored in a storage medium (e.g., ROM/RAM, diskette, optical disk) that includes instructions for causing a terminal (which may be a cell phone, computer, server, air conditioner, network device, etc.) to perform the methods described in the various embodiments of the present application.

Embodiments of the present application have been described above with reference to the accompanying drawings, but the present application is not limited to the specific embodiments described above, which are merely illustrative and not restrictive, and those skilled in the art, from the enlightenment of this application, may make many forms falling within the protection of this application without departing from the spirit and claimed scope of the invention.

1. A method for evaluating brittleness of a deep shale reservoir, characterized by comprising:
determining a Rickman brittleness index BI_{Rickman} of the deep shale reservoir;
determining an effective pressure Pe of the deep shale reservoir according to a pore pressure Pₚ and an overlying formation pressure P of the deep shale reservoir; and
adjusting BI_{Rickman} to obtain BI according to an exponential relationship of the brittleness index BI with Pe of the deep shale reservoir.

2. The method for evaluating the brittleness of the deep shale reservoir according to claim 1, wherein the determining an effective pressure Pe of the deep shale reservoir according to a pore pressure Pₚ and an overlying formation pressure P of the deep shale reservoir comprises:
determining the overlying formation pressure P of the deep shale reservoir according to P=∫ₜ_{₀}^{t}ρ(t)V(t)gdt, wherein g is gravity acceleration, ρ(t) is measured rock density, V(t) is measured rock velocity, t is time depth of the deep shale reservoir, and t₀ is time depth of a reference datum plane;
determining a hydrostatic pressure P₀ of the deep shale reservoir according to P₀=ρ_{w}gH, wherein H is formation burial depth, and ρ_{w} is formation water density;
determining a pore pressure Pₚ of the deep shale reservoir according to the Eaton method, wherein,
$${P}_{p} = P - ( P - {P}_{0} ) {( \frac{Δ {t}_{n}}{Δ {t}_{s}} )}^{c} ,$$
c is the Eaton coefficient, Δtₛ is measured interval transit time, and Δtₙ is normal compaction interval transit time; and
determining the effective pressure Pe of the deep shale reservoir according to Pe=P−Pₚ.

3. The method for evaluating the brittleness of the deep shale reservoir according to claim 2, further comprising:
determining Δtₙ according to Δtₙ=Δtₘ+(Δtₘₗ−Δtₘ)e^{-aH}, wherein Δtₘ is the interval transit time of rock matrix, Δtₘₗ is the interval transit time of the surface or seabed, H is the formation buried depth, and a is the regional index.

4. The method for evaluating the brittleness of the deep shale reservoir according to claim 1, wherein the adjusting BI_{Rickman} to obtain BI according to an exponential relationship of the brittleness index BI with Pe of the deep shale reservoir comprises:
determining BI as follows:
BI=B_{Rickman}[e^{m(40-Pe)}−n]/l;
wherein, l is amplitude modulation factor, and m and n are obtained by fitting laboratory rock core stress-strain analysis data.

5. The method for evaluating the brittleness of the deep shale reservoir according to claim 4, wherein the determining an effective pressure Pe of the deep shale reservoir according to a pore pressure Pₚ and an overlying formation pressure P of the deep shale reservoir comprises:
determining the overlying formation pressure P of the deep shale reservoir according to P=∫ₜ_{₀}^{t}ρ(t)V(t)gdt, wherein g is gravity acceleration, ρ(t) is measured rock density, V(t) is measured rock velocity, t is time depth of the deep shale reservoir, and t₀ is time depth of a reference datum plane;
determining a hydrostatic pressure P₀ of the deep shale reservoir according to P₀=ρ_{w}gH, wherein H is formation burial depth, and ρ_{w} is formation water density;
determining a pore pressure Pₚ of the deep shale reservoir according to the Eaton method, wherein,
$${P}_{p} = P - ( P - {P}_{0} ) {( \frac{Δ {t}_{n}}{Δ {t}_{s}} )}^{c} ,$$
c is the Eaton coefficient, Δtₛ is measured interval transit time, and Δtₙ is normal compaction interval transit time; and
determining the effective pressure Pe of the deep shale reservoir according to Pe=P−Pₚ.

6. The method for evaluating the brittleness of the deep shale reservoir according to claim 5, further comprising:
determining Δtₙ according to Δtₙ=Δtₘ+(Δtₘₗ−Δtₘ)e^{-aH}, wherein Δtₘ is the interval transit time of rock matrix, Δtₘₗ is the interval transit time of the surface or seabed, H is the formation buried depth, and a is the regional index.

7. A computer device comprising:
a memory, a processor, and a computer program stored on the memory and being operable on the processor;
wherein the computer program, when executed by the processor, performs the steps of the method for evaluating the brittleness of the deep shale reservoir of claim 1.

8. A computer device comprising:
a memory, a processor, and a computer program stored on the memory and being operable on the processor;
wherein the computer program, when executed by the processor, performs the steps of the method for evaluating the brittleness of the deep shale reservoir of claim 2.

9. A computer device comprising:
a memory, a processor, and a computer program stored on the memory and being operable on the processor;
wherein the computer program, when executed by the processor, performs the steps of the method for evaluating the brittleness of the deep shale reservoir of claim 3.

10. A computer device comprising:
a memory, a processor, and a computer program stored on the memory and being operable on the processor;
wherein the computer program, when executed by the processor, performs the steps of the method for evaluating the brittleness of the deep shale reservoir of claim 4.

11. A computer device comprising:
a memory, a processor, and a computer program stored on the memory and being operable on the processor;
wherein the computer program, when executed by the processor, performs the steps of the method for evaluating the brittleness of the deep shale reservoir of claim 5.

12. A computer device comprising:
a memory, a processor, and a computer program stored on the memory and being operable on the processor;
wherein the computer program, when executed by the processor, performs the steps of the method for evaluating the brittleness of the deep shale reservoir of claim 6.

13. A computer readable storage medium having stored thereon a brittleness evaluation program for a deep shale reservoir which, when executed by a processor, performs the steps of the method for evaluating the brittleness of the deep shale reservoir of claim 1.

14. A computer readable storage medium having stored thereon a brittleness evaluation program for a deep shale reservoir which, when executed by a processor, performs the steps of the method for evaluating the brittleness of the deep shale reservoir of claim 2.

15. A computer readable storage medium having stored thereon a brittleness evaluation program for a deep shale reservoir which, when executed by a processor, performs the steps of the method for evaluating the brittleness of the deep shale reservoir of claim 3.

16. A computer readable storage medium having stored thereon a brittleness evaluation program for a deep shale reservoir which, when executed by a processor, performs the steps of the method for evaluating the brittleness of the deep shale reservoir of claim 4.

17. A computer readable storage medium having stored thereon a brittleness evaluation program for a deep shale reservoir which, when executed by a processor, performs the steps of the method for evaluating the brittleness of the deep shale reservoir of claim 5.

18. A computer readable storage medium having stored thereon a brittleness evaluation program for a deep shale reservoir which, when executed by a processor, performs the steps of the method for evaluating the brittleness of the deep shale reservoir of claim 6.
